(19) United States
(12) Patent Application Publication
(10) Pub. No.: US 2026/0004898 A1
(43) Pub. Date: Jan. 1, 2026

(54) DYNAMIC PROCESSING OF GENETIC ORDERS FOR PATIENTS

(71) Applicant: Helix, Inc., San Mateo, CA (US)

(72) Inventors: Zubin Madon, Palo Alto, CA (US); Nathaniel Angeles, Foster City, CA (US); Kirti Sapra, Cincinnati, OH (US); Igor Wolff, Orlando, FL (US); Anna Zhu, Seattle, WA (US); Jose Urtasun, Santiago (CL)

(21) Appl. No.: 18/755,301

(22) Filed: Jun. 26, 2024

(51) Int. Cl. G16H 10/60; G16H 40/20; G16H 80/00

(52) U.S. Cl. CPC G16H 10/60; G16H 40/20; G16H 80/00

(57) ABSTRACT

System and method of genetic testing. In an embodiment, a system receives one or more incoming medical messages from a requestor regarding a patient, and determines whether a data repository stores sequencing data for the patient. When sequencing data is stored for the patient, the system parses an incoming medical message of the incoming medical messages to identify a procedure code indicating a genetic test requested for the patient, processes the procedure code to select a workflow to analyze the sequencing data with one or more analysis tools for the genetic test, and initiates the workflow to analyze the sequencing data for the patient and generate test results. The system then provides the test results for the patient to the requestor.

### TECHNICAL FIELD

The following disclosure relates to the field of medicine, and more particularly, to genetic testing for patients.

### BACKGROUND

A physician, clinician, or the like, such as in treatment of a patient, may want to order some type of genomic or genetic testing for the patient. Genetic testing comprises the analysis of genetic material for an individual, such as a gene, a part or group of genes, or the entire genome. For example, a physician that diagnoses a condition of a patient may order a genetic test to confirm or rule out the diagnosis based on gene variants or alleles identified for the patient and those known to be associated with the condition. However, orders for genetic testing (referred to generally herein as genetic orders) can be difficult and inconvenient for a physician, which may dissuade them from requesting a genetic test even though the resulting information may be beneficial in the treatment of the patient.

### SUMMARY

Embodiments described herein provide an automated solution for handling genomic or genetic orders. As a general overview, an apparatus referred to as a genomic data system, is configured to process medical messages received from a requestor, such as a system/device operated by a physician, requesting a genetic test for a patient. The genomic data system is configured to extract a procedure code indicating the requested genetic test, and dynamically select a workflow to analyze sequencing data for the patient (if existing). The genomic data system is configured to respond to the requestor with test results for the patient, which may be used in diagnosis or treatment of the patient. One technical benefit is the genomic data system is able to dynamically select which analysis tool(s) is used to analyze sequencing data for the patient based on the received procedure code. Thus, the requestor is tasked with providing a desired procedure code in a medical message, which is a straightforward and convenient way to submit a genetic order. A requestor may therefore be motivated to order genetic testing for patients, which may improve the quality of care.

In an embodiment, a system comprises a data repository configured to store sequencing data for sequencing participants generated based on genomic sequencing, and a test manager, comprising a processor and memory, configured to execute an algorithm to receive one or more incoming medical messages from a requestor regarding a patient, and determine whether the data repository stores sequencing data for the patient. When the data repository stores the sequencing data for the patient, the test manager is configured to execute the algorithm to parse an incoming medical message of the incoming medical messages to identify a procedure code indicating a genetic test requested for the patient, process the procedure code to select a workflow to analyze the sequencing data with one or more analysis tools for the genetic test, and initiate the workflow to analyze the sequencing data for the patient and generate test results. The test manager is configured to provide the test results for the patient to the requestor.

In an embodiment, a method comprises storing sequencing data for sequencing participants generated based on genomic sequencing, receiving one or more incoming medical messages from a requestor regarding a patient, and determining whether sequencing data is stored for the patient. When the sequencing data is stored for the patient, the method further comprises parsing an incoming medical message of the incoming medical messages to identify a procedure code indicating a genetic test requested for the patient, processing the procedure code to select a workflow to analyze the sequencing data with one or more analysis tools for the genetic test, and initiating the workflow to analyze the sequencing data for the patient and generate test results. The method further comprises providing the test results for the patient to the requestor.

Other embodiments may include computer readable media, other systems, or other methods as described below.

The above summary provides a basic understanding of some aspects of the specification. This summary is not an extensive overview of the specification. It is intended to neither identify key or critical elements of the specification nor delineate any scope particular embodiments of the specification, or any scope of the claims. Its sole purpose is to present some concepts of the specification in a simplified form as a prelude to the more detailed description that is presented later.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 is a block diagram of a bioinformatics system in an illustrative embodiment.

FIG. 2 is a block diagram illustrating genetic testing in an illustrative embodiment.

FIG. 3 is a flow chart illustrating a method of genetic testing in an illustrative embodiment.

FIG. 4 is a block diagram of a genomic data system in an illustrative embodiment.

FIG. 5A is a block diagram illustrating data analysis resources in an illustrative embodiment.

FIG. 5B is a flow chart illustrating a method of performing data analysis of raw sequence data in an illustrative embodiment.

FIG. 6 is a block diagram illustrating workflows in an illustrative embodiment.

FIGS. 7A-7F are block diagrams illustrating medical messaging in illustrative embodiments.

FIG. 8 is a flow chart illustrating a method of providing a genomics service in an illustrative embodiment.

FIG. 9A is a diagram illustrating a code mapping of procedure codes to workflows in an illustrative embodiment.

FIG. 9B is a diagram illustrating a genetic test in an illustrative embodiment.

FIG. 10 is a block diagram illustrating classification of a patient in an illustrative embodiment.

FIG. 11 is a diagram illustrating communicative coupling of a genomic data system with other systems/servers in an illustrative embodiment.

FIG. 12 is a flow chart illustrating a method of providing a genomics service in an illustrative embodiment.

FIG. 13 is a flow chart illustrating a method of aggregating procedure codes in an illustrative embodiment.

FIG. 14 is a diagram illustrating a genomic data system handling multiple procedure codes in an illustrative embodiment.

FIGS. 15-17 are block diagrams illustrating a display on a requestor client in illustrative embodiments.

FIGS. 18A-18B are block diagrams illustrating medical messaging in illustrative embodiments.

### DETAILED DESCRIPTION

The figures and the following description illustrate specific exemplary embodiments. It will thus be appreciated that those skilled in the art will be able to devise various arrangements that, although not explicitly described or shown herein, embody the principles of the embodiments and are included within the scope of the embodiments. Furthermore, any examples described herein are intended to aid in understanding the principles of the embodiments, and are to be construed as being without limitation to such specifically recited examples and conditions. As a result, the inventive concept(s) is not limited to the specific embodiments or examples described below, but by the claims and their equivalents.

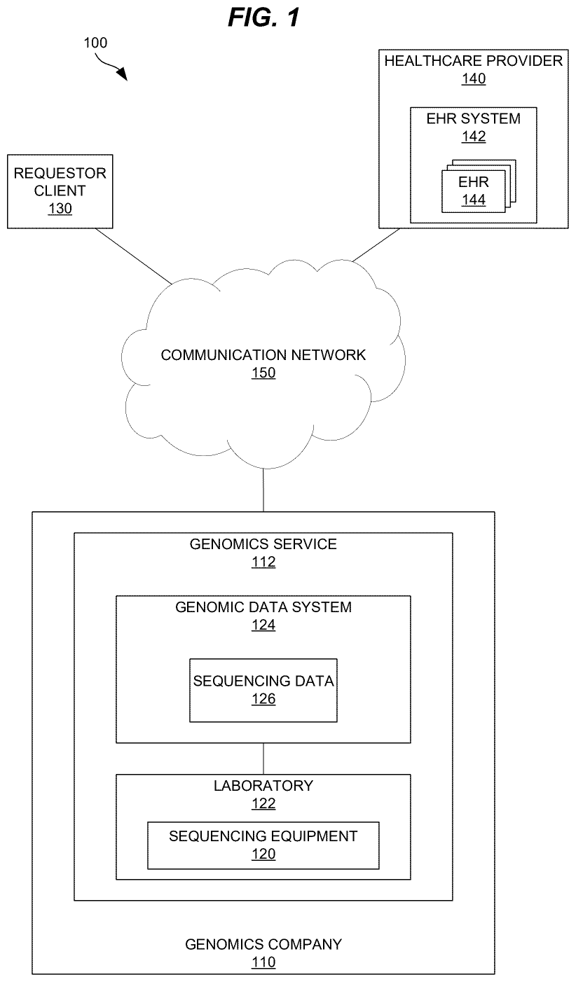

FIG. 1 is a block diagram of a bioinformatics system 100 in an illustrative embodiment. Bioinformatics is a scientific field related to the development or application of tools or applications to analyze and interpret biological data, such as DNA (deoxyribonucleic acid) sequences. At a high level, bioinformatics system 100 comprises any combination of systems, components, devices and/or computer technology to collect, store, and/or analyze genomic data. In an embodiment, bioinformatics system 100 includes a genomics service 112 offered by a genomics company 110. For the genomics service 112, genomics company 110 may perform or offer sample collection, DNA (deoxyribonucleic acid) or genomic sequencing, secure data storage of the sequencing data generated by sequencing processes, analysis of the sequencing data, etc. The genomics service 112 may be a fee-based service, such as a subscription-based service where a subscription is obtained to receive the genomics service 112.

For a sequencing process, genomics company 110 may implement or use sequencing equipment 120 (e.g., a sequencing instrument(s), a sequencing platform, a next-generation sequencing (NGS) platform, etc.) at a laboratory 122 or the like, which is configured to perform a sequencing process on biological samples. For example, DNA sequencing is a process of determining an exact sequence of nucleotides, or bases, in a DNA molecule. Sequencing equipment 120 may therefore include a DNA sequencer and/or other instruments configured to determine the order of the four bases: G (guanine), C (cytosine), A (adenine), and T (thymine). Genomic sequencing is a process of determining the entire genetic makeup of an organism.

Genomics company 110 may further implement a genomic data system 124 configured to store (i.e., secure data storage) sequencing data 126 (also referred to as genomic sequencing data), analyze sequencing data 126, and/or otherwise manage sequencing data 126. For example, genomic data system 124 may process the sequencing data 126 (e.g., raw sequence data) to identify variants or alleles (i.e., variant calling). The sequencing data 126 as described herein may include raw DNA or genomic sequences (e.g., order of the bases), and any associated data extracted from the raw sequences, such as aligned sequence data, variant information or variant call data, etc. Genomic data system 124 may be implemented at a laboratory 122 of the genomics company 110, such as on servers or other on-premises resources at the laboratory 122. Alternatively, genomic data system 124 may be implemented on one or more external platforms, such as a cloud infrastructure of a cloud computing platform. Cloud computing is the delivery of computing resources, including storage, processing power, databases, networking, analytics, artificial intelligence, and software applications, over an internet connection. Some examples of a cloud computing platform may comprise Amazon Web Services (AWS), Google Cloud, Microsoft Azure, etc. Further, although genomics company 110 is illustrated as implementing sequencing equipment 120 and genomic data system 124, it is understood that the sequencing equipment 120 and genomic data system 124 may be distributed among different companies, entities, platforms, etc.

Genomic data system 124 is configured to manage genetic testing for patients in response to data communications received from a requestor, such as a requestor client 130, an EHR system 142 of a healthcare provider 140, etc. Genomic data system 124 is configured to communicate with external systems or devices via a communication network 150. Communication network 150 may comprise a Wide Area Network (WAN), such as the Internet, a telecommunications network, an enterprise network or private network, a Wireless Local Area Network (WLAN), etc., or any combination thereof. For example, genomic data system 124 may be configured to communicate with requestor client 130 via communication network 150 to exchange medical messaging or other data communications. In another example, genomic data system 124 may be configured to communicate with EHR system 142 of healthcare provider 140 to exchange medical messaging or other data communications. A healthcare provider 140 is a licensed person or organization that provides healthcare services. A healthcare provider 140 implements an EHR system 142 that maintains, tracks, and/or stores EHR data 144 for a plurality of patients. An Electronic Health Record (EHR) is an electronic or digital version of a patient's medical history maintained by a healthcare provider. EHR data 144 may include patient care information, including demographics (e.g., date of birth, gender, ethnicity, blood type, income, postal code, etc.), progress notes, problems, medications, vital signs, past medical history, immunizations, laboratory data or test results, radiology reports, etc.

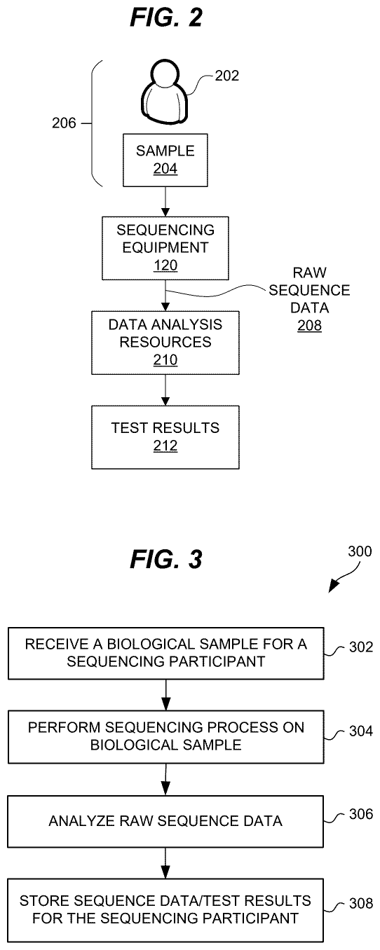

FIG. 2 is a block diagram illustrating genetic testing in an illustrative embodiment. FIG. 3 is a flow chart illustrating a method 300 of genetic testing in an illustrative embodiment. The steps of the flow charts described herein are not all inclusive and may include other steps not shown, and the steps may be performed in an alternative order. A biological sample 204 (e.g., blood, saliva, etc.) of an individual 202 is received at laboratory 122 for sequencing (step 302). An individual 202 that volunteers or consents to genomic sequencing of a biological sample 204 is referred to as a sequencing participant 206. The sequencing equipment 120 at laboratory 122 performs a sequencing process on the biological sample 204 to generate raw sequence data 208 associated with the sequencing participant 206 (step 304). Data analysis resources 210 may then analyze or otherwise process the raw sequence data 208, such as alignment, variant calling, and/or any other analysis (step 306). The analysis process generates test results 212 (also referred to as diagnostic results, analysis results, genomic analysis results, analysis output, etc.). The raw sequence data 208 and any data or information generated by the analysis of the raw sequence data 208, such as the aligned sequence data, variant information or variant call data, etc., may be collectively referred to as sequencing data 126 for, or associated with, a sequencing participant 206. The sequencing data 126 may comprise data for a whole genome, a subset of the genes that make up a genome, etc. The sequencing data 126 and/or test results 212 are stored in secure data storage (step 308), such as in a data repository.

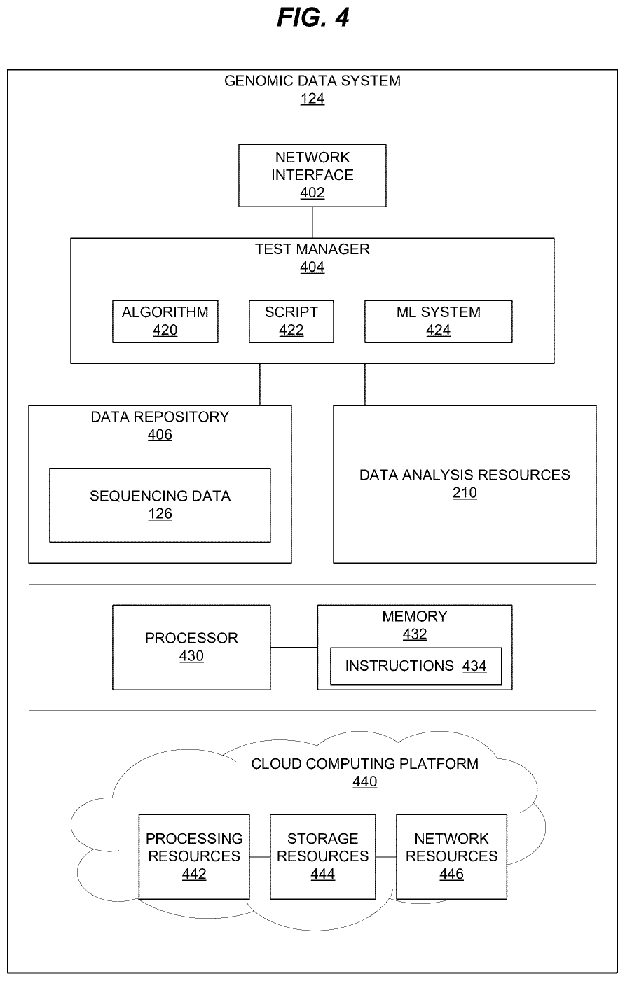

FIG. 4 is a block diagram of genomic data system 124 in an illustrative embodiment. Genomic data system 124 may include the following subsystems: a network interface component 402, a test manager 404, a data repository 406, and data analysis resources 210 that operate on one or more platforms. Network interface component 402 may comprise circuitry, logic, hardware, means, etc., configured to exchange messages, documents, and/or electronic data communications with external devices or systems. Network interface component 402 may operate using a variety of protocols and/or Application Programming Interfaces (APIs). Test manager 404 may comprise circuitry, logic, hardware, means, etc., configured to manage the storage, processing, analysis, etc., of sequencing data 126. For example, test manager 404 may be configured to identify, select, and/or generate workflows for the storage, processing, analysis, etc., of sequencing data 126. Test manager 404 may execute an algorithm(s) 420, such as a script(s) 422, a machine learning (ML) system(s) 424, etc., to perform one or more actions or tasks as described herein.

Data repository 406 comprises secure data storage configured to store sequencing data 126. Data analysis resources 210 are configured to perform data analysis on raw sequence data 208 and/or other sequencing data 126. Data analysis resources 210 may comprise internal or local resources of genomic data system 124, and/or may comprise one or more external analysis tools/applications.

One or more of the subsystems of genomic data system 124 may be implemented on a hardware platform comprised of analog and/or digital circuitry. For example, network interface component 402, test manager 404, and/or one or more data analysis resources 210 may be implemented on one or more processors 430 that execute instructions 434 (i.e., computer readable code) for software that are loaded into memory 432. A processor 430 comprises an integrated hardware circuit configured to execute instructions 434 to provide the functions of genomic data system 124. Processor 430 may comprise a set of one or more processors or may comprise a multi-processor core, depending on the particular implementation. Memory 432 is a non-transitory computer readable storage medium for data, instructions, applications, etc., and is accessible by processor 430. Memory 432 is a hardware storage device capable of storing information on a temporary basis and/or a permanent basis. Memory 432 may comprise a random-access memory, or any other volatile or non-volatile storage device.

One or more of the subsystems of genomic data system 124 may be implemented on cloud computing platform 440 (e.g., AWS) or another type of processing platform. Cloud resources may be provisioned on cloud computing platform 440, such as processing resources 442 (e.g., physical or hardware processors, a server, a virtual server or virtual machine (VM), a virtual central processing unit (vCPU), etc.), storage resources 444 (e.g., physical or hardware storage, virtual storage, etc.), and/or networking resources 446, although other resources are considered herein. Genomic data system 124 may be built upon the provisioned resources with instructions, programming, code, etc. For example, network interface component 402 may be provisioned on networking resources 446, test manager 404 and/or one or more data analysis resources 210 may be provisioned on processing resources 442, and data repository 406 may be provisioned on storage resources 444.

Genomic data system 124 may include various other components not specifically illustrated in FIG. 4.

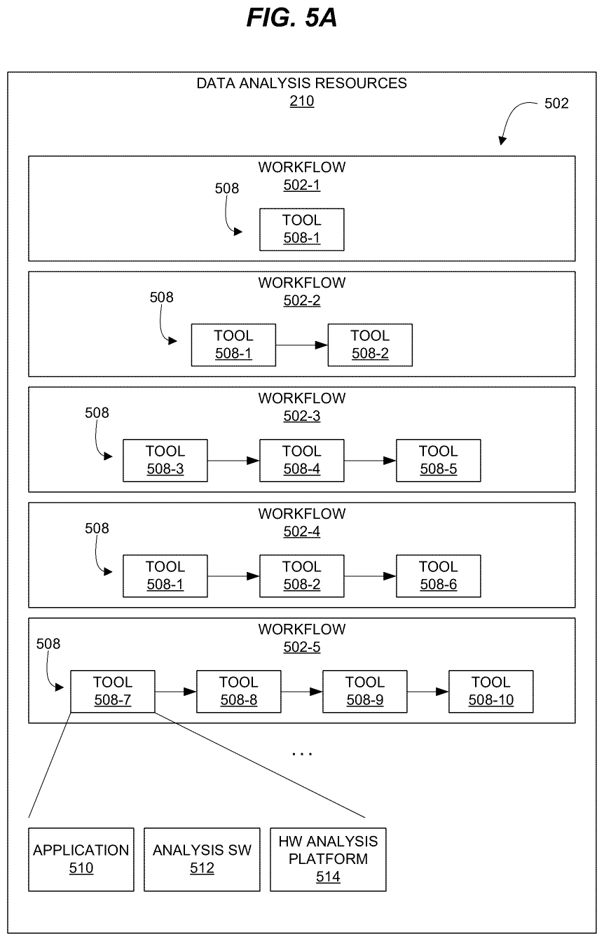

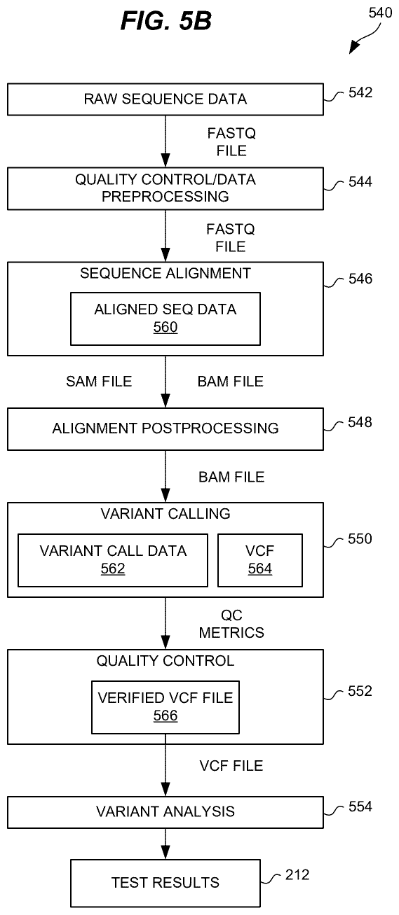

FIG. 5A is a block diagram illustrating data analysis resources 210 in an illustrative embodiment. Data analysis resources 210 may support a plurality of workflows 502 (e.g., workflows 502-1, 502-2, 502-3, etc.), which may also be referred to as analysis pipelines, that are configured to perform genetic testing on sequencing data 126. A workflow 502 (or a portion of a workflow) comprises a set of one or more data processing elements that receives sequencing data 126 as input, and outputs test results 212 for a genetic test. For example, a workflow 502 may include one or more analysis tools 508 (e.g., analysis tools 508-1, 508-2, 508-3, etc.). An analysis tool 508 is configured to extract insights from sequencing data 126. An analysis tool 508 may comprise an application 510, analysis software 512, a hardware analysis platform 514, etc. Each workflow 502 may process the sequencing data 126 differently to produce test results 212. Test manager 404, as in FIG. 4, may select a workflow 502 based on a local policy or criteria, based on an order or request for a genetic test, etc.

FIG. 5B is a flow chart illustrating a method 540 of performing data analysis of raw sequence data 208 in an illustrative embodiment. To begin, raw sequence data 208 is received (step 542), such as from sequencing equipment 120. The raw sequence data 208 may be received in a file encoded in a standard file format, such as a FASTQ file. The raw sequence data 208 may contain biases and/or complex artifacts depending on the platform used for base calling. Thus, one or more analysis tools 508 may perform quality control (QC) and/or data preprocessing on the raw sequence data 208 (step 544). For quality control, for example, an analysis tool 508 may generate summary statistics assessing the overall quality of the raw sequence data 208. An analysis tool 508 may preprocess the raw sequence data 208 to remove reads (i.e., sequences corresponding to all or part of a single DNA fragment) having quality scores below a quality threshold, remove adapter sequences, remove sequences with fewer than a threshold number of bases, etc.

An analysis tool 508 may then perform sequence alignment on the (preprocessed) raw sequence data 208 (step 546). Sequence alignment is a process of mapping the sequences to a reference genome or reference sequences. The analysis tool 508 that performs sequence alignment may output aligned sequence data 560, such as a sequence alignment map (SAM) file or a binary alignment map (BAM) file. An analysis tool 508 may also perform quality control (QC) of the BAM file, such as to evaluate key sequencing metrics, verify sufficient sequencing coverage was achieved, detect evidence of contamination, etc.

An analysis tool 508 may then perform alignment postprocessing on the aligned sequence data (step 548). Sequence alignments may be processed to detect and correct incorrect alignments in order to minimize artifacts in the downstream analyses. An analysis tool 508 may then perform variant calling on the aligned sequence data 560 (step 550). Variant calling is a process of identifying differences between a sequence and the reference sequence. The variants may include single nucleotide polymorphisms (SNPs), insertions/deletions (indels), and structural variations. The analysis tool 508 that performs variant calling outputs variant call data 562 in a Variant Call Format (VCF) file 564, for example, which is a type of text file format containing variation data (i.e., indicating the variants).

An analysis tool 508 may then perform quality control on QC metrics and/or other data associated with the VCF file 564 to generate a verified VCF file 566 (step 552). For example, the analysis tool 508 may remove false positives from the initial variant data set. The analysis tool 508 may compare the initial variant data set to certain metrics (e.g., probabilistic likelihood or unlikelihood of certain variant data).

An analysis tool 508 may then perform analysis on the variants identified in the verified VCF file 566 (step 554). The analysis may depend on a particular genetic test that was ordered. For example, the analysis tool 508 may perform analysis for a diagnostic test, a predictive test, a pharmacogenomic test, and/or another type of genetic test. The analysis tool 508 outputs test results 212 based on the analysis. The test results 212, verified VCF file 566, FASTQ files, SAM files, BAM files, and any other electronic data files may be stored in data repository 406. Although data analysis was described above in terms of variant calling, other types of data analysis are considered herein for genetic tests.

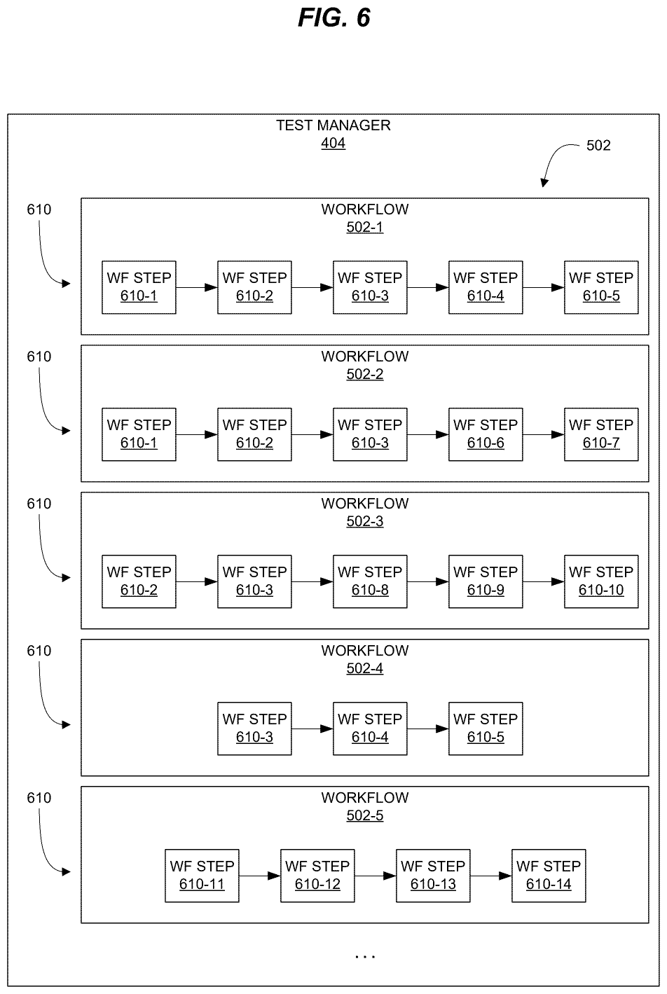

In an embodiment, genomic data system 124 is configured to dynamically select a workflow 502 for a genetic test ordered or requested by a requestor. The workflow 502 selected for a genetic test may include data analysis resources 210 as described above in FIG. 5A, and may include other workflow steps, such as communicating with the requestor, sample acquisition (if needed), sequencing, etc. FIG. 6 is a block diagram illustrating workflows 502 in an illustrative embodiment. Test manager 404 may support one or multiple workflows 502 related to genetic testing. Each workflow 502 comprises a series or sequence of actions or workflow steps 610 (e.g., workflow step 610-1, 610-2, 610-3, etc.) that define a job or how work should be done regarding a genetic test. For example, one or more workflow steps 610 may be defined to confirm or acquire consent to sequencing from a patient. One or more workflow steps 610 may be defined to order or acquire a sample 204 from a patient. One or more workflow steps 610 may be defined to initiate or perform a sequencing process on a sample 204, such as at sequencing equipment 120. One or more workflow steps 610 may be defined to initiate or perform analysis of sequencing data 126 (e.g., raw sequence data 208), such as with one or more analysis tools 508 (see FIG. 5A). One or more workflow steps 610 may be defined to initiate or perform review of data generated via data analysis.

In an embodiment, genomic data system 124 is configured to exchange medical messages with a requestor regarding genetic testing, such as requestor client 130 or EHR system 142 as shown in FIG. 1, or another type of requesting party or entity. Medical messaging (also referred to as clinical messaging, healthcare messaging, etc.) is the exchange of electronic messages in the medical or clinical domain. For example, medical messaging may be utilized by a physician to convey instructions to third parties, such as laboratories, imaging centers, pharmacies, and/or other groups. In an embodiment, medical messages may be in plain text (e.g., unencrypted) with any Protected Health Information (PHI) anonymized to be HIPAA-compliant. In another embodiment, some or all of the medical messages may be encrypted or otherwise security-protected.

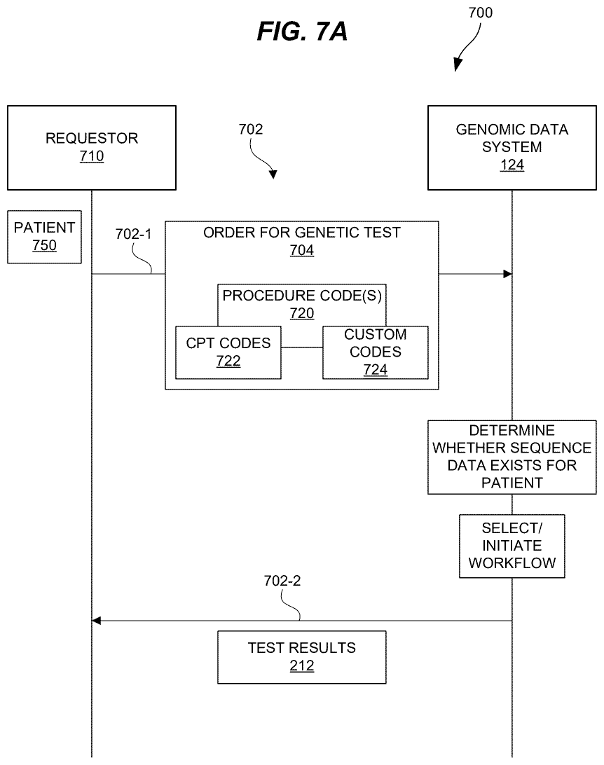

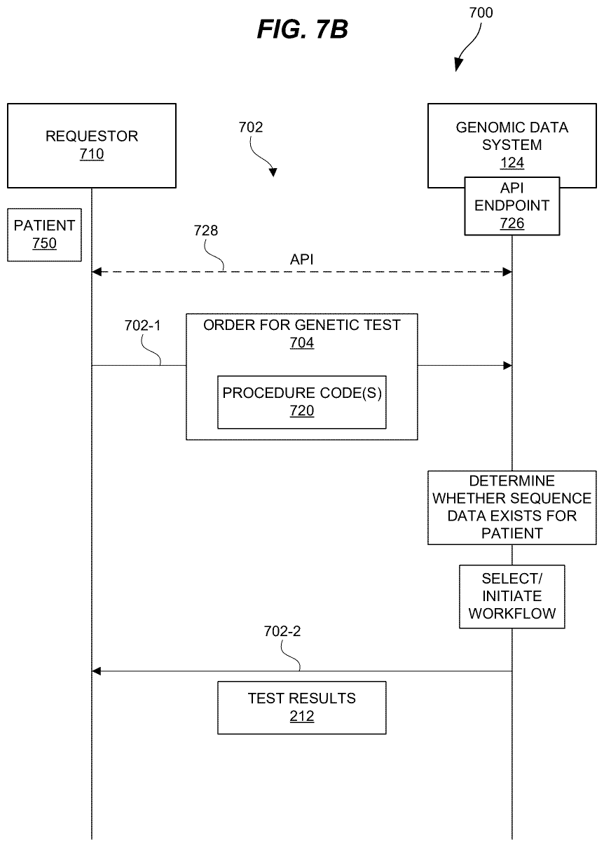

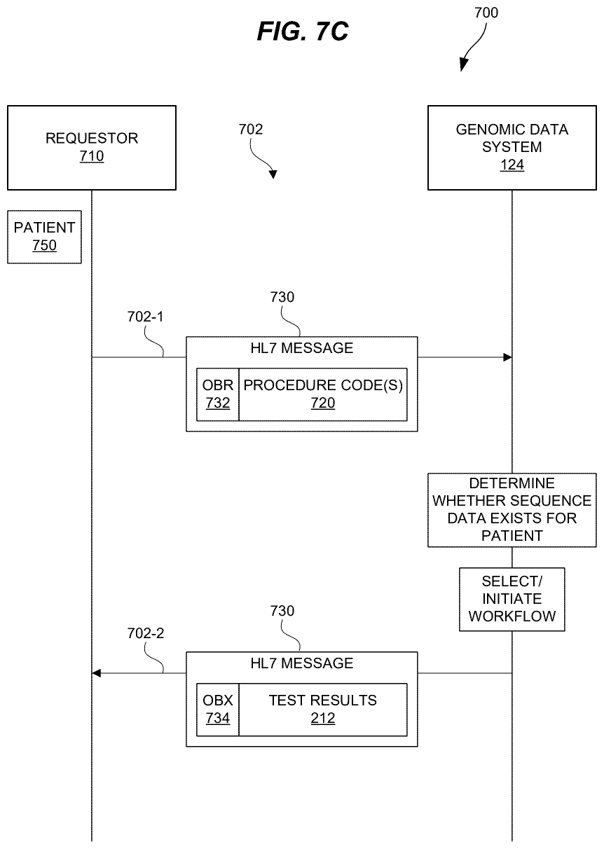

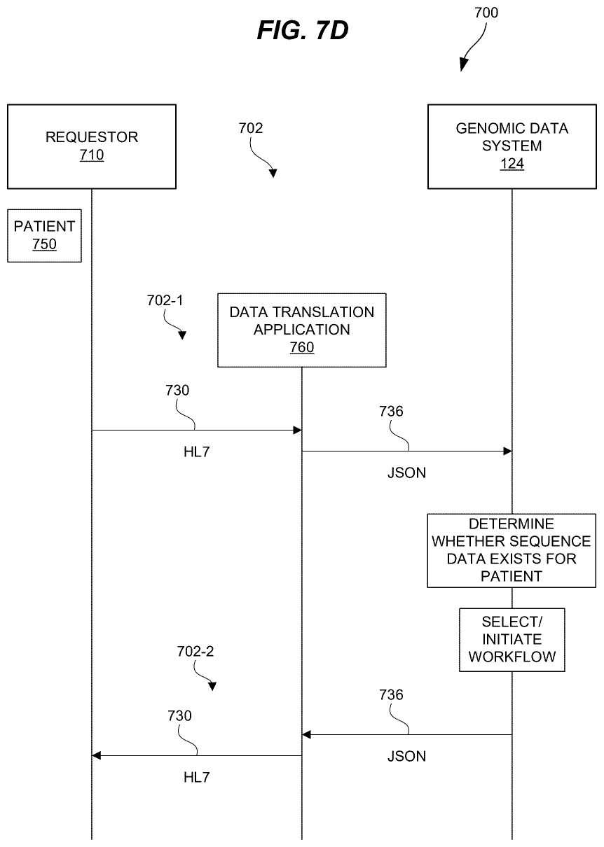

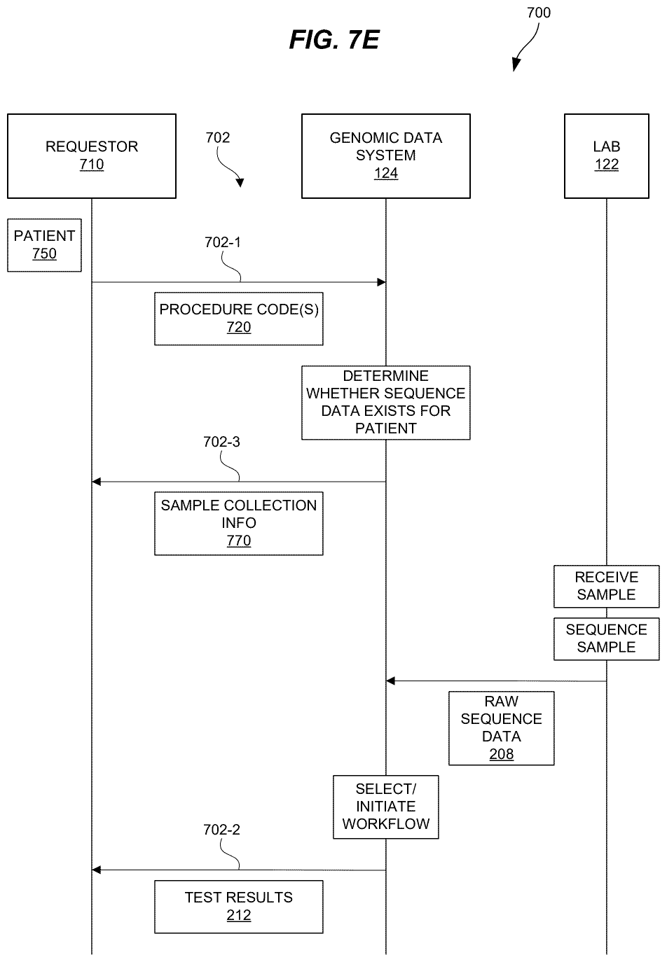

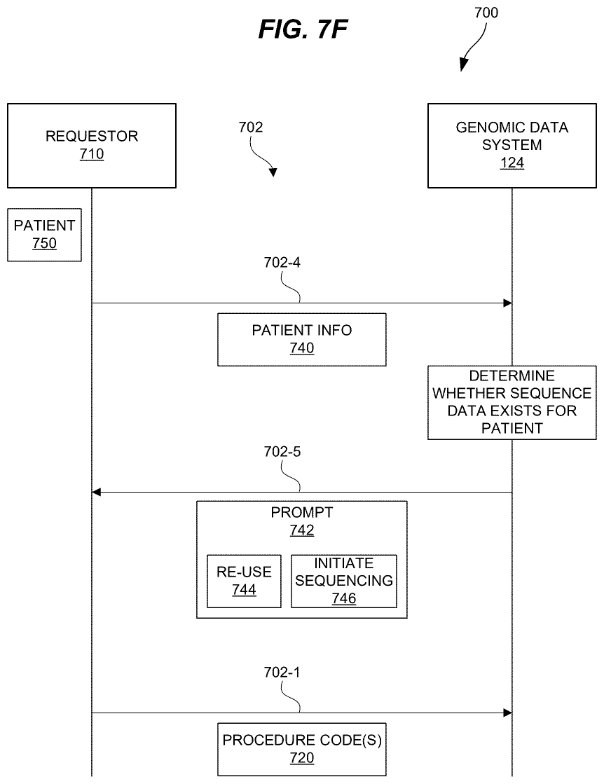

FIGS. 7A-7F are block diagrams illustrating medical messaging 700 in illustrative embodiments. In general, medical messaging 700 comprises the exchange of one or more medical messages 702 between a requestor 710 and genomic data system 124 (e.g., through network interface component 402), such as over a communication network 150. A medical message 702 comprises an electronic data communication that transfers or conveys electronic or digital data over a communication channel, a tunnel, etc. A medical message 702 may be in the form of a request, a response, a query, a file, etc. The format of a medical message 702 may vary as desired. Medical messaging 700 and/or actions performed in FIGS. 7A-7F are provided as an example, and other messaging/actions may be performed in other embodiments. Also, the order of messages/actions provided in FIGS. 7A-7F may be performed in an alternate order. Any reference to “first”, “second”, etc., herein is used to distinguish different elements and not necessarily meant to indicate any particular order.

In FIG. 7A, genomic data system 124 and requestor 710 exchange medical messages 702 regarding a patient 750, such as via communication network 150. Genomic data system 124 receives a medical message 702-1 from requestor 710 regarding the patient 750. Medical messages 702 received from a requestor 710 may be referred to as first medical messages, incoming medical messages, etc. A medical message 702-1 received from requestor 710 may include an order 704 or request for a genetic test. Thus, medical message 702-1 may include or contain one or more procedure codes 720 that indicates or specifies a genetic test requested or ordered for a patient 750. In an embodiment, a procedure code 720 may comprise a Current Procedural Terminology (CPT®) code 722, which provides a uniform nomenclature for coding medical procedures and services. One technical benefit is CPT codes 722 streamline reporting, and increase accuracy and efficiency. In an embodiment, a procedure code 720 may comprise a custom code 724 specified in genomic data system 124 or in the genomics service 112 for a genetic test. One technical benefit is a custom code 724 may be mapped to a specific type of genetic test, which allows genomic data system 124 to efficiently and reliably select a workflow 502 to perform.

In response to medical message 702-1 (or a prior medical message 702 from requestor 710), genomic data system 124 determines whether the patient 750 was previously sequenced. In embodiments described herein, the patient 750 under consideration may have previously been a sequencing participant 206, and sequencing data 126 may exist and be stored for the patient 750, such as in data repository 406 or another, accessible data repository (e.g., an external repository). Alternatively, the patient 750 under consideration may not have previously been a sequencing participant 206, and no sequencing data 126 exists or is stored for the patient 750. When the patient 750 was previously sequenced, for example, genomic data system 124 processes the procedure code 720 to identify or select a workflow 502 to analyze the sequencing data 126 with one or more analysis tools 508 for the genetic test. Genomic data system 124 initiates the selected workflow 502 to analyze the sequencing data 126 for the patient 750 and generate test results 212. Genomic data system 124 then sends, transmits, or provides a medical message 702-2 to the requestor 710 indicating the test results 212. Medical messages 702 sent to the requestor 710 may be referred to as second medical messages, outgoing medical messages, etc. One technical benefit is genomic data system 124 may automatically and immediately initiate a selected workflow 502 on existing sequencing data 126 for the patient 750 so that test results 212 may be provided to the requestor 710 in a timely manner (e.g., in one minute, five minutes, ten minutes, etc.).

In FIG. 7B, genomic data system 124 may implement an API 728 for the medical messaging 700, which defines how the requestor 710 and genomic data system 124 communicate with each other, such as using requests and responses. Genomic data system 124 may therefore specify an API endpoint 726, which is a digital location where an API 728 receives requests about a specific resource, such as a Uniform Resource Locator (URL). One technical benefit is the API 728 in genomic data system 124 may consume data from several independent sources without requiring the sources to develop their own specialized applications.

In FIG. 7C, genomic data system 124 may exchange Health Level Seven (HL7®) messages 730 with requestor 710. HL7 is a standard for exchanging information between medical information systems, and is an example of a medical messaging format. Medical message 702-1 may therefore comprise an HL7 message 730 that contains a procedure code(s) 720 for a requested genomics test regarding a patient 750. Likewise, medical message 702-2 may comprise an HL7 message 730 that contains the test results 212 generated for the requested genomics test. In general, the format of an HL7 message 730 includes segments, fields, components, and subcomponents. The procedure codes 720 may be inserted in an observation request segment 732 (OBR), for example, and genomic data system 124 may be configured to parse the observation request segment 732 to extract the procedure code(s) 720 from an HL7 message 730. The test results 212 may be inserted in an observation result segment 734 (OBX), for example, and genomic data system 124 may be configured to insert the test results 212 in the observation result segment 734 of an HL7 message 730. However, other segments, fields, components, etc., of an HL7 message may be used. One technical benefit is HL7 format is standardized within the medical domain, and many systems (e.g., EHR systems 142) are programmed to exchange information via HL7.

In FIG. 7D, a data translation application 760 may be implemented between genomic data system 124 and requestor 710. A data translation application 760 normalizes data between systems to allow for interoperability between the systems that may use different protocols, formats, etc. One example of data translation application 760 is a Redox platform. In this embodiment, requestor 710 may send an HL7 message 730 to data translation application 760 containing a procedure code(s) 720, and data translation application 760 may convert or translate the HL7 message 730 into a JavaScript Object Notation (JSON) file 736 or another type of message or file. Data translation application 760 may then send the JSON file 736 containing the procedure code(s) 720 to genomic data system 124. Genomic data system 124 may therefore be configured to parse the JSON file 736 to extract the procedure code(s) 720. Further, genomic data system 124 may be configured to insert the test results 212 in a JSON file 736, and send the JSON file 736 to data translation application 760. In turn, data translation application 760 may convert or translate the JSON file 736 into an HL7 message 730 that is sent to the requestor 710 with the test results 212. One technical benefit is the genomic data system 124 may be able to communicate with different types of requestors 710 that use different systems without having to be programmed according to the specifications for each of the different systems.

In FIG. 7E, genomic data system 124 again receives a medical message 702-1 from requestor 710 regarding a patient 750, which may include or contain one or more procedure codes 720 associated with patient 750. In response to the medical message 702-1 (or a prior medical message 702 from requestor 710), genomic data system 124 determines whether the patient 750 was previously sequenced. When the patient 750 was not previously sequenced, for example, genomic data system 124 sends, transmits, or provides a medical message 702-3 to requestor 710 with sample collection information 770. The sample collection information 770 includes a request for a sample 204 from the patient 750, instructions for collecting the sample 204, etc.

When a sample 204 from the patient 750 is subsequently received at the laboratory 122, genomic sequencing is performed on the sample 204 by the sequencing equipment 120. Genomic data system 124 receives raw sequence data 208 for the patient 750, and processes the procedure code 720 to identify or select a workflow 502 to analyze the raw sequence data 208 with one or more analysis tools 508 for the genetic test. Genomic data system 124 initiates the workflow 502 to analyze the raw sequence data 208 for the patient 750 and generate test results 212. Genomic data system 124 then sends, transmits, or provides a medical message 702-2 to the requestor 710 indicating the test results 212. One technical benefit is genomic data system 124 may automatically initiate a selected workflow 502 on newly-generated raw sequence data 208 for the patient 750 so that the test results 212 may be provided to the requestor 710.

In FIG. 7F, genomic data system 124 and requestor 710 may exchange medical messages 702 prior to genomic data system 124 receiving a procedure code(s) 720 from requestor 710. In an embodiment, genomic data system 124 receives a medical message 702-4 from requestor 710 regarding a patient 750. Medical message 702-4 includes or contains patient data or patient information 740 for the patient 750. The patient information 740 comprises non-individually identifiable information for the patient 750, such as an anonymized identifier. In response to medical message 702-4, genomic data system 124 determines whether the patient 750 was previously sequenced. Genomic data system 124 then generates a prompt 742 for the requestor 710, and sends, transmits, or provides the prompt 742 to the requestor 710 in a medical message 702-5. In an embodiment, the patient 750 may be previously sequenced, and the prompt 742 may include a selection option to re-use 744 the raw sequence data 208 existing for the patient 750 in a genomics test(s). In an embodiment, the patient 750 may not be previously sequenced, and the prompt 742 may include a selection option to initiate sequencing 746 for the patient 750. It is understood that the prompt 742 may include other information or selection options as desired. Requestor 710 then orders a genomics test(s) based on the prompt 742 from genomic data system 124. One technical benefit is genomic data system 124 indicates whether raw sequence data 208 exists for the patient 750 before a genomics test(s) is ordered so the requestor 710 is able to make an informed decision regarding a genomics test(s), such as timeliness.

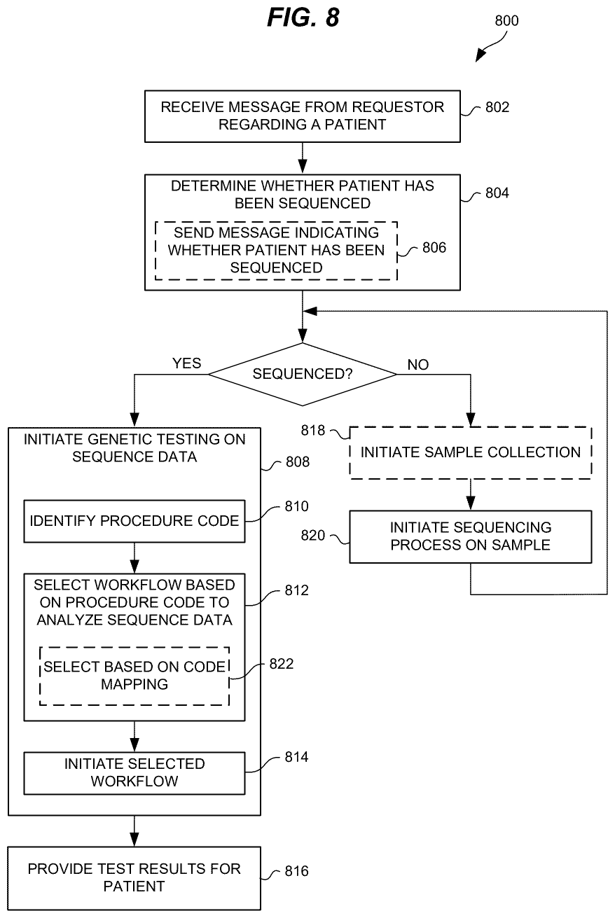

FIG. 8 is a flow chart illustrating a method 800 of providing a genomics service in an illustrative embodiment. The steps of method 800 will be described with reference to genomic data system 124 in FIG. 4, but those skilled in the art will appreciate that method 800 may be performed in other systems or devices.

Genomic data system 124 receives one or more medical messages 702 from a requestor 710 regarding a patient 750 (step 802). In response to a medical message 702, genomic data system 124 determines whether the patient 750 has been sequenced (step 804). For example, genomic data system 124 may determine whether (valid) sequencing data 126 (e.g., raw sequence data 208) is stored for the patient 750, such as in data repository 406. Genomic data system 124 may also send a medical message 702 to the requestor 710 indicating whether the patient 750 has been sequenced (optional step 806).

When the patient 750 has been sequenced, genomic data system 124 initiates genetic testing on the sequencing data 126 (step 808). To do so, genomic data system 124 identifies a procedure code 720 indicating a genetic test requested or ordered for the patient 750 (step 810). For example, genomic data system 124 may parse a medical message 702 to identify or extract a procedure code 720 for the requested genetic test. Genomic data system 124 processes the procedure code 720 to select a workflow 502 to analyze the sequencing data 126 with one or more analysis tools 508 for the genetic test (step 812). Genomic data system 124 then initiates the selected workflow 502 to analyze the sequencing data 126 for the patient 750 and generate test results 212 (step 814). Genomic data system 124 sends, transmits, or otherwise provides the test results 212 to the requestor 710 in a medical message 702 (step 816). For example, genomic data system 124 may send a medical message 702 to the requestor 710 with the test results 212, may update EHR data 144 for the patient 750, etc.

When the patient 750 has not been sequenced, genomic data system 124 may take alternative steps to get the patient 750 sequenced. For example, patient 750 may not have provided a sample 204 to laboratory 122 for sequencing. Thus, genomic data system 124 may initiate sample collection for the patient 750 (optional step 818). To do so, genomic data system 124 may send a request (i.e., a medical message 702) to the patient 750, healthcare provider 140, etc., to provide a sample 204 of genetic material. If/when a sample 204 is received for the patient 750, genomic data system 124 may initiate a sequencing process on the sample 204 using sequencing equipment 120 (step 820). As described above, sequencing equipment 120 generates raw sequence data 208 for the patient 750, which may be stored in data repository 406. Genomic data system 124 may then initiate genetic testing on the raw sequence data 208 (step 808). One technical benefit is genomic data system 124 may call the analysis tools 508 of the selected workflow 502 (e.g., immediately) when the patient 750 has already been sequenced so that test results 212 may be provided to the requestor 710 in a timely manner. Alternatively, genomic data system 124 may perform actions to get the patient 750 sequenced so that genetic testing may be performed.

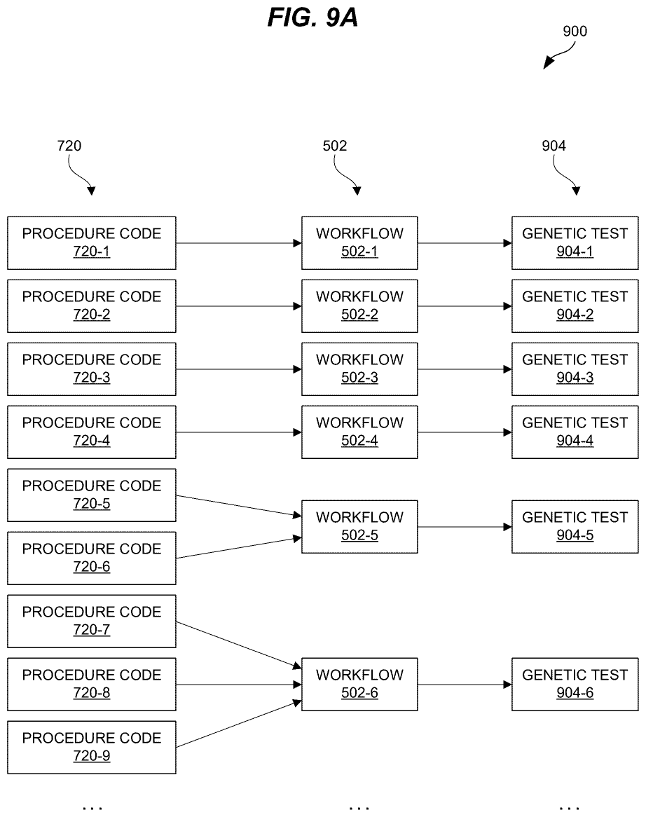

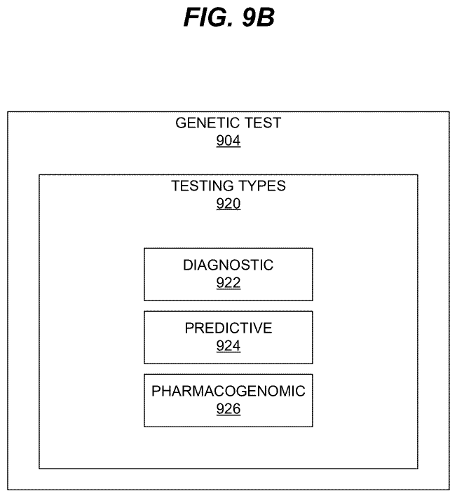

In an embodiment, genomic data system 124 may store a code mapping of procedure codes 720 to workflows 502 so that a desired genetic test is performed in response to a procedure code 720 provided in a medical message 702. FIG. 9A is a diagram illustrating a code mapping 900 of procedure codes 720 to workflows 502 in an illustrative embodiment. A plurality of procedure codes 720 (e.g., procedure codes 720-1, 720-2, 720-3, etc.) may be defined for genomic data system 124. As described above, the procedure codes 720 may be CPT codes 722, may be custom codes 724, such as predefined codes or internal codes, etc. The procedure codes 720 are mapped to workflows 502 (e.g., workflow 502-1, 502-2, 502-3, etc.) according to the code mapping 900. There may be a one-to-one mapping of a procedure code 720 to a workflow 502, a many-to-one mapping of multiple procedure codes 720 to a workflow 502, or other types of mapping. Each workflow 502, in turn, is configured to perform (at least) a genetic test 904 (e.g., genetic tests 904-1, 904-2, 904-3, etc.) on sequencing data 126 using one or more analysis tools 508. A genetic test 904 is a test performed on a genome or part(s) of a genome of a human. Results of a genetic test 904 are test results 212 that are reported to a requestor 710, for example. Genomic data system 124 may use the code mapping 900 to select a workflow 502 (see optional step 822 of FIG. 8). For example, when receiving a medical message 702 from the requestor 710 that includes a procedure code 720 indicating a genetic test 904 requested or ordered for the patient 750, genomic data system 124 may use the code mapping 900 using the procedure code 720 as an index to identify a workflow 502 mapped to the procedure code 720. Genomic data system 124 may then analyze the sequencing data 126 with one or more analysis tools 508 of the selected workflow 502.

FIG. 9B is a diagram illustrating a genetic test 904 in an illustrative embodiment. A genetic test 904 may be of different testing types 920. One testing type 920 is a diagnostic test 922, which is used to confirm or rule out a condition or disease of a patient. For example, a physician or clinician may diagnose a patient with a particular genetic condition based on relevant symptoms, and a diagnostic test 922 may be ordered to confirm an initial diagnosis, or to rule out a possible cause of the symptoms. Another testing type 920 may be a predictive test 924 (also referred to as screening), which is used determine whether or not a patient is susceptible to a particular condition before displaying any symptoms. For example, a physician or clinician may order a predictive test 924 when a genetic condition has been diagnosed in a family member. Another testing type 920 may be a pharmacogenomic test 926, which is used determine the response of a patient to drugs or medication based on the patient's genome. For example, before prescribing a mediation to a patient, a physician or clinician may order a pharmacogenomic test 926 to determine whether a particular medicine will be effective or ineffective, whether the medicine is likely to cause side-effects, whether the medicine is likely to cause an adverse reaction, etc. Although some examples of testing types 920 are provided in FIG. 9B, other testing types 920 may be considered herein.

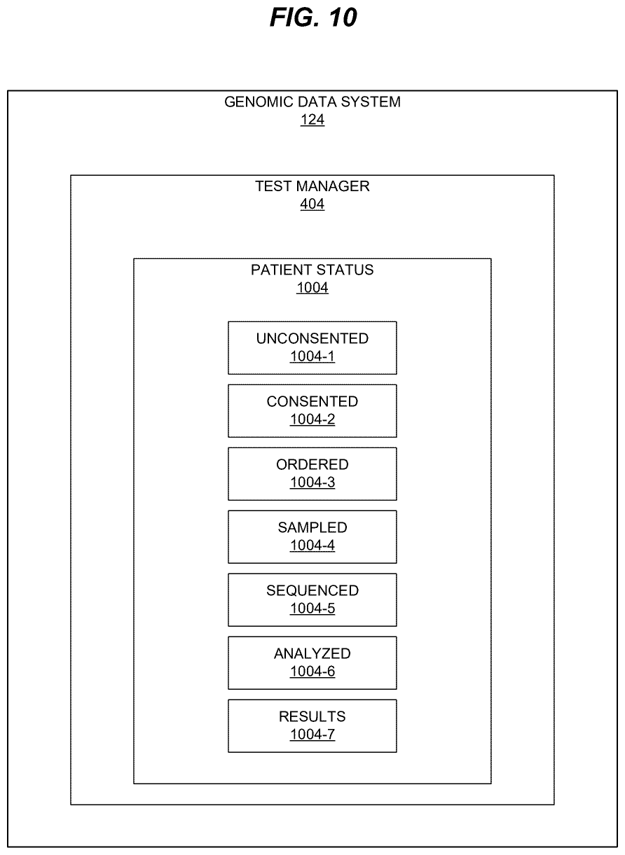

In an embodiment, genomic data system 124 may categorize or classify the status of a patient 750 in response to a medical message 702 from a requestor 710. FIG. 10 is a block diagram illustrating classification of a patient 750 in an illustrative embodiment. Genomic data system 124 may initiate a classification process, such as in response to a medical message 702, to classify a state or status of the patient 750. For example, the patient status 1004 may include an unconsented status 1004-1, where a patient 750 has no record of consent to sequencing. The patient status 1004 may include a consented status 1004-2, where a patient 750 has an associated record of consent, but no associated sample or sample kit has been received or registered at the laboratory 122 for sampling. The patient status 1004 may include an ordered status 1004-3, where a patient 750 has received a kit for sampling, but has not provided the kit or a sample to the laboratory 122. The patient status 1004 may include a sampled status 1004-4, where a kit or sample for a patient 750 has been received and/or registered at the laboratory 122, but has not been sequenced. The patient status 1004 may include a sequenced status 1004-5, where raw sequence data 208 (e.g., FASTQ data) has been generated for a patient 750, but analysis of the raw sequence data 208 has not been performed. The patient status 1004 may include an analyzed status 1004-6, where raw sequence data 208 of a patient 750 has been analyzed (e.g., raw sequence data 208 has been aligned (e.g., in a BAM file) and/or called (e.g., in a VCF file)), but no formal test results 212 have been generated or verified. The patient status 1004 may include a results status 1004-7, where test results 212 (e.g., formal reports) have been prepared for a patient 750 (e.g., based on aligned and called sequencing data).

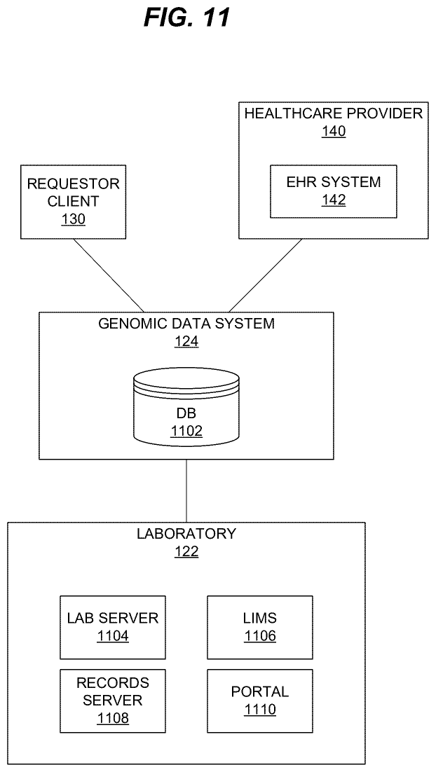

For the classification process and/or a genomics service 112 in general, genomic data system 124 may implement or communicate with a variety of other systems/servers. FIG. 11 is a diagram illustrating communicative coupling of genomic data system 124 with other systems/servers in an illustrative embodiment. As described in FIG. 1, genomic data system 124 may be communicatively coupled (e.g., via a communication network 150) to a requestor client 130 and/or EHR system 142. Genomic data system 124 may implement or communicate with a database 1102 or the like that stores information regarding individuals, such as sequencing participants 206 and/or patients 750 yet to be sequenced. Genomic data system 124 may implement or communicate with a lab server 1104 (e.g., of laboratory 122) that stores information regarding sequencing processes (e.g., analysis tools 508 used for a sequencing process), a Laboratory Information Management System (LIMS) 1106 configured to manage samples 204 and associated data, a records server 1108 configured to store records for patients 750 or sequencing participants 206, a portal 1110 configured to interact with sequencing participants 206 or potential sequencing participants, etc. Genomic data system 124 may communicate with other systems/servers not shown.

The classification process may comprise reviewing or processing data stored in database 1102, and maintaining or updating a patient status 1004 for each patient 750. The patient status 1004 may be updated live or in real-time based on information received, such as receipt of consents (e.g., electronic consent) at a server (e.g., lab server 1104, records server 1108, etc.), internal reports from LIMS 1106, etc.

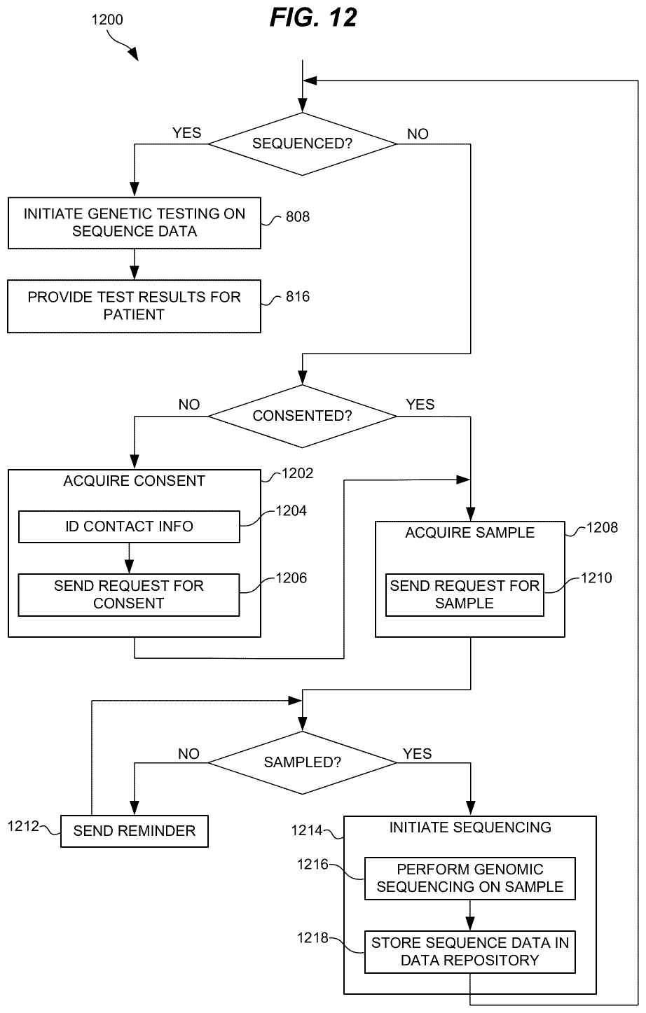

FIG. 12 is a flow chart illustrating a method 1200 of providing a genomics service 112 in an illustrative embodiment. As described above, genomic data system 124 is configured to determine whether a patient 750 under consideration has already been sequenced (see step 804 of FIG. 8). For example, genomic data system 124 may parse data repository 406, query lab server 1104 and/or records server 1108, etc., to determine whether a patient 750 has been sequenced, and valid raw sequence data 208 is stored for the patient 750. When the patient 750 has been sequenced, genomic data system 124 classifies the patient 750 as having a sequenced status 1004-5. When the patient 750 has a sequenced status 1004-5, genomic data system 124 may initiate genetic testing on the raw sequence data 208 for the patient 750 (step 808 of FIG. 8) by identifying a procedure code 720 indicating a genetic test 904 requested for the patient 750, selecting a workflow 502 to analyze the raw sequence data 208 with one or more analysis tools 508 for the genetic test 904, and initiating the workflow 502 to analyze the raw sequence data 208 for the patient 750. As part of the selected workflow 502, one or more analysis tools 508 may run to analyze the raw sequence data 208, such as to perform alignment and/or variant calling (e.g., generate a VCF file)). At this point, genomic data system 124 may transition the patient 750 to an analyzed status 1004-6. When the patient 750 has an analyzed status 1004-6 and as part of the selected workflow 502, one or more analysis tools 508 may run based on the indicated procedure code 720 to generate test results 212. For example, an analysis tool 508 may run for analysis of cardiac genomics in relation to a procedure asking for a cardigan genetic test. Some procedure codes 720 may further require validation by a genomics counselor before results may be sent to a patient 750. Upon validation, genomic data system 124 may generate a report comprising the test results 212, such as an electronic document classifying one or more variants as benign, pathogenic, or of unknown significance. Genomic data system 124 may then transition the patient 750 a results status 1004-7. When the patient 750 has a results status 1004-7, genomic data system 124 may send, transmit, or otherwise provide the test results 212 to the requestor 710 (step 816 in FIG. 8).

In an embodiment, when the patient 750 has been sequenced and has an analyzed status 1004-6, aligned sequence data, variant information or variant call data, etc., has already been generated for the patient 750. Thus, when genomic data system 124 receives a request for a patient 750 having an analyzed status 1004-6, genomic data system 124 may initiate genetic testing on this sequencing data 126 (e.g., aligned sequence data or variant call data) for the patient 750 by identifying a procedure code 720 indicating a genetic test 904 requested for the patient 750, selecting a workflow 502 to analyze the sequencing data 126 with one or more analysis tools 508 for the genetic test 904, and initiating the workflow 502 to analyze the sequencing data 126 for the patient 750.

In an embodiment, when genomic data system 124 receives a request for a patient 750 having a results status 1004-7, genomic data system 124 may determine whether or not the analysis tool(s) 508 used for the patient 750 has been updated. If so, genomic data system 124 may transition the patient 750 to a sequenced status 1004-5 or analyzed status 1004-6, and re-run analysis with the updated analysis tool(s) 508. Alternatively, if a request for a patient 750 relates to a condition that requires specialized analysis tools 508 that have not yet been run for the patient 750, genomic data system 124 may transition the patient 750 to a sequenced status 1004-5 or analyzed status 1004-6, and select a new workflow 502 for processing via whatever specialized analysis tools 508 are required.

When the patient 750 has not been sequenced, genomic data system 124 may initiate sample collection for the patient 750 (see step 816 in FIG. 8). For example, genomic data system 124 may determine whether the patient 750 under consideration has consented to sequencing (e.g., as indicated by reference to records server 1108 of laboratory 122). When the patient 750 has not consented to sequencing, genomic data system 124 classifies the patient 750 as having an unconsented status 1004-1. When the patient 750 has an unconsented status 1004-1, genomic data system 124 may acquire consent from the patient 750 (step 1202). For example, genomic data system 124 may select a workflow 502 or otherwise perform actions/operations to acquire consent from the patient 750. Genomic data system 124 may identify contact information for the patient 750 (step 1204), such as by parsing the received medical message 702, querying an EHR system 142 of a healthcare provider 140 that sourced the medical message 702, and/or querying database 1102, records server 1108, etc. Genomic data system 124 may send a request to the patient 750 to consent to genomic data collection (step 1206), before proceeding further with that patient 750. The request may comprise, for example, a link to a portal 1110 (e.g. a registration portal) for a records server 1108. Upon receiving the consent, genomic data system 124 may transition the patient 750 to a consented status 1004-2.

When the patient 750 has a consented status 1004-2 (e.g., as indicated by reference to a records server 1108, and confirmation with LIMS 1106 that a sampling kit has not been prepared for the patient 750), genomic data system 124 may acquire a sample 204 from the patient 750 (step 1208). For example, genomic data system 124 may send a request to the patient 750, healthcare provider 140, etc., to provide a sample 204 of genetic material (step 1210). If the healthcare provider 140, for example, prefers collection within a medical facility, genomic data system 124 may further transmit a request for collection of the genetic material by the patient 750 at the medical facility. Alternatively, genomic data system 124 may order a drop-shipping service to send a sampling kit directly to the patient 750 for self-sampling. After the sampling kit has been ordered (e.g., via drop-shipping or referral to a medical facility), genomic data system 124 may transition the patient 750 to an ordered status 1004-3.

When the patient 750 has an ordered status 1004-3, genomic data system 124 determines whether a sample has been received for the patient 750 under consideration. This may be determined, for example, by subscribing to notifications or otherwise monitoring patient identifiers on received sampling kits, and identifying laboratory identifiers for those patients 750. Genomic data system 124 may wait a configurable time period for receipt of a sample 204 of the patient 750 (e.g., a completed sampling kit at the laboratory 122 that corresponds with the patient 750). In an embodiment, if a patient 750 having an ordered status 1004-3 takes longer than a threshold amount of time to provide a sample 204, genomic data system 124 may send a reminder to the patient 750 and/or healthcare provider 140 for that patient 750 (step 1212). After a sample 204 has been received (e.g., that passes quality control standards), genomic data system 124 may transition the patient 750 to a sampled status 1004-4.

When the patient 750 has a sampled status 1004-4, genomic data system 124 may initiate a sequencing process on the sample 204 (step 1214), which corresponds with step 820 of FIG. 8. For example, sequencing equipment 120 may perform genomic sequencing on the sample 204 to generate raw sequence data 208 (step 1216). Genomic data system 124 may then store raw sequence data 208 in data repository 406 (step 1218). After sequencing is completed, genomic data system 124 may transition the patient 750 to a sequenced status 1004-5. One technical benefit is genomic data system 124 is able to determine different statuses of a patient 750 regarding genetic testing, and perform actions and/or dynamically select different workflows 502 based on the current status of the patient 750.

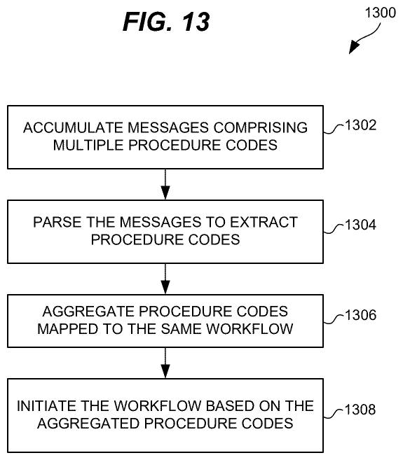

In an embodiment, multiple procedure codes 720 may be received for a patient 750 in a single medical message 702 or multiple medical messages 702. Thus, genomic data system 124 may be configured to perform an aggregation process. FIG. 13 is a flow chart illustrating a method 1300 of aggregating procedure codes 720 in an illustrative embodiment. The steps of method 1300 will be described with reference to genomic data system 124 in FIG. 4, but those skilled in the art will appreciate that method 1300 may be performed in other systems or devices.

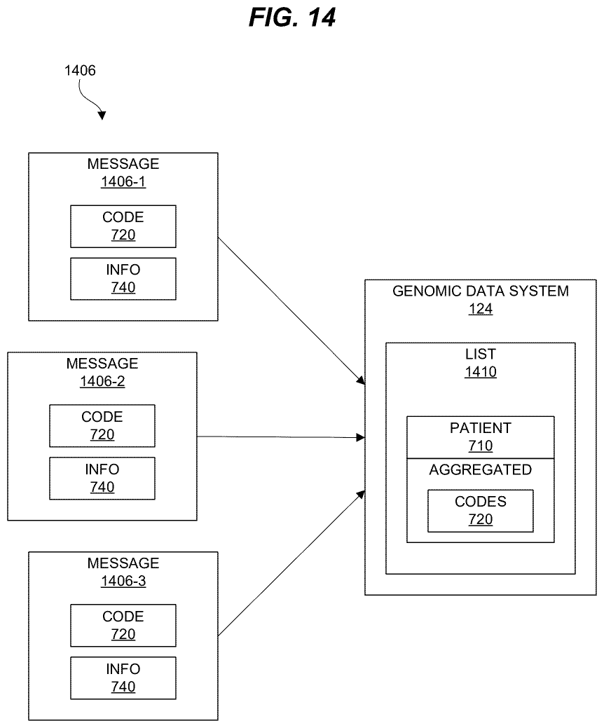

Method 1300 represents an aggregation process, where genomic data system 124 receives and/or accumulates one or more medical messages 702 comprising multiple procedure codes 720 (step 1302). FIG. 14 is a diagram illustrating genomic data system 124 handling multiple procedure codes 720 in an illustrative embodiment. Medical messages 702 are illustrated generally as messages 1406 (e.g., message 1406-1, message 1406-2, and message 1406-3), which may comprise HL7 messages 730, JSON files 736, and/or messages in another format. Each message 1406 includes one or more procedure codes 720, and may also include patient information 740. Genomic data system 124 may accumulate messages 1406 for one or more patients 750 over a period of time, such as over the course of several minutes, an hour, a day, etc.

Genomic data system 124 parses the medical messages 702 to extract the procedure codes 720 (step 1304 of FIG. 13) and other information, such as patient information 740. After accumulating and processing the messages 1406, genomic data system 124 generates a list 1410 of patients 750 and corresponding procedure codes 720 for those patients 750. Genomic data system 124 maintains multiple analysis tools 508 that are each capable of handling one or more procedure codes 720. Procedures codes 720 may be mapped to workflows 502 on a many-to-many basis, but in general, many procedure codes 720 may be mapped to a single workflow 502 used for genetic testing. For example, a single workflow 502 for performing variant calling for cardiac-related genes may be mapped to tens or hundreds of procedure codes 720 that each request genetic testing regarding a single variant related to cardiac health.

Genomic data system 124 aggregates the procedure codes 720 that are mapped to the same workflow 502 (step 1306). Genomic data system 124 then initiates the workflow 502 to analyze the sequencing data 126 for the patient 750 based on the aggregated procedure codes 720 and generates test results 212 (step 1308). One technical benefit is the workflow 502 runs once for the aggregated procedure codes 720, which is advantageous as the workflow 502 may be processing-intensive. In an embodiment, whenever a new message 1406 is received with a procedure code 720 that maps to the workflow 502 (which has already run for the patient 750), the test results 212 of the workflow 502 may be retrieved without re-running the workflow 502.

### Example

In the following example, additional processes, systems, and methods may be described in the context of genetic testing. The processes, systems, and methods described in this example may be incorporated in embodiments described above as desired.

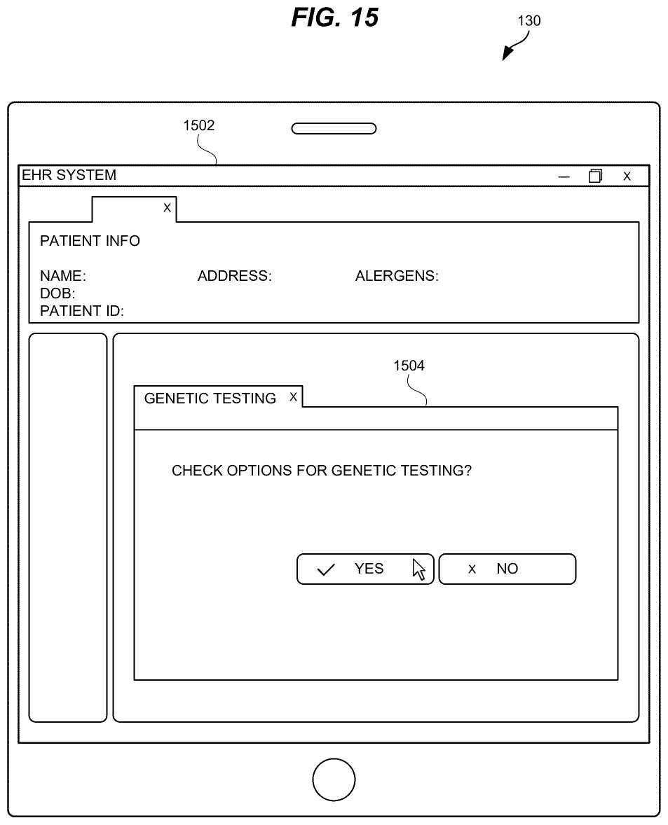

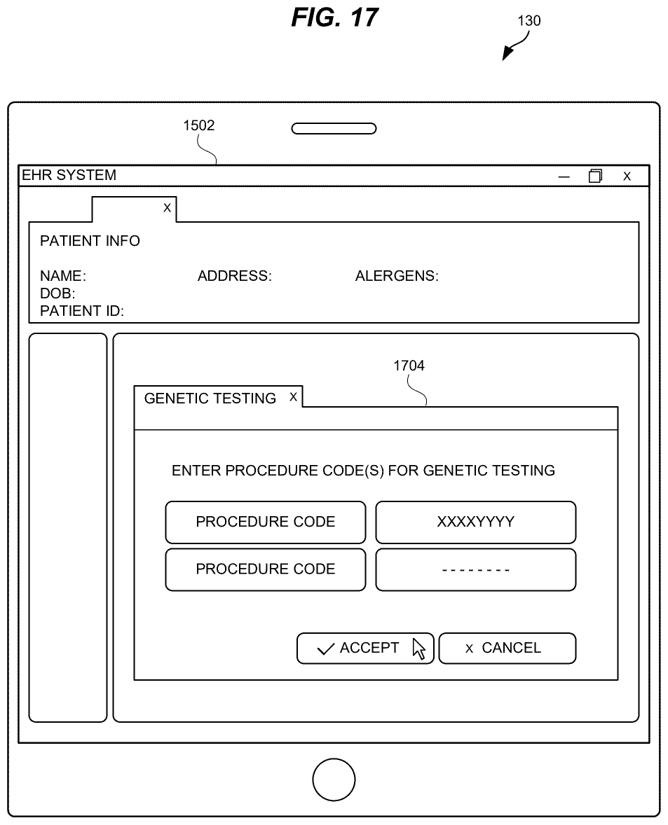

FIGS. 15-17 are block diagrams illustrating a display 1502 on a requestor client 130 in illustrative embodiments. Requestor client 130 is configured to access or run an application for an EHR system 142. The application provides a GUI that displays graphical elements, windows, screens, etc., to interact with a physician. For example, the GUI may display a genetic testing screen 1504 allowing the physician to check for options regarding genetic testing for a patient, as shown in FIG. 15.

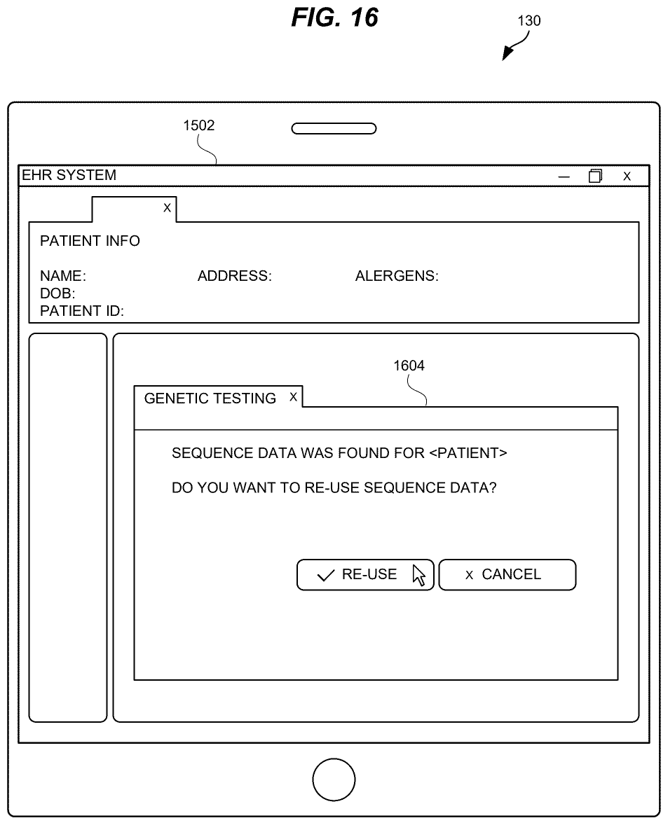

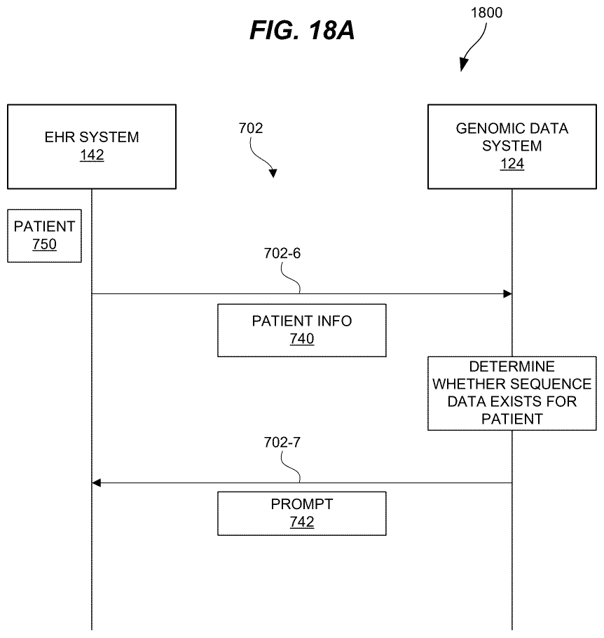

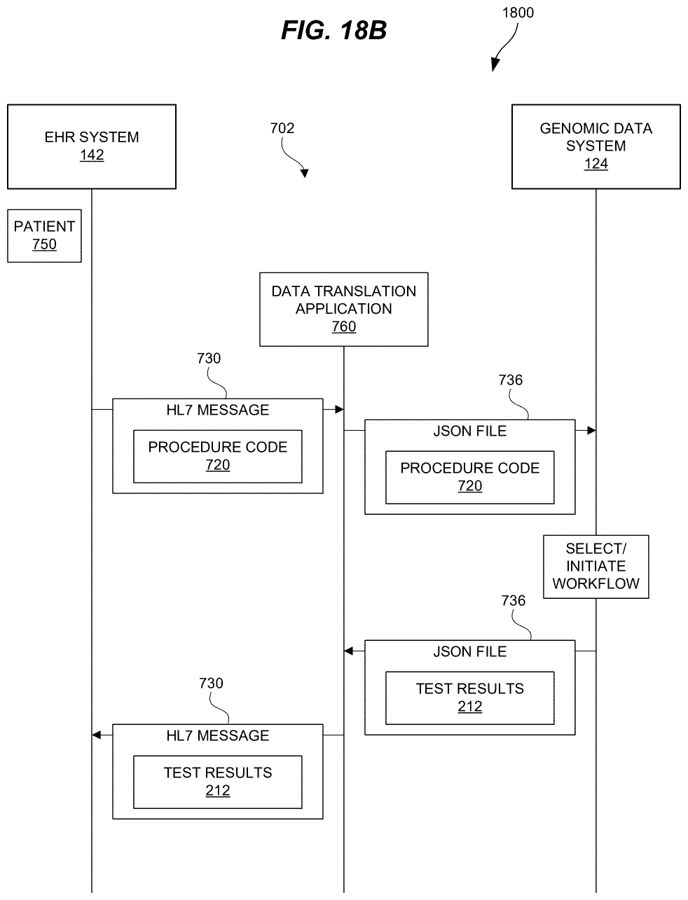

FIGS. 18A-18B are block diagrams illustrating medical messaging 1800 in illustrative embodiments. When the physician selects “YES” in FIG. 15, EHR system 142 sends a medical message 702-6 to genomic data system 124 with patient information 740 so that genomic data system 124 is able to identify the patient 750 under consideration (see FIG. 18A). In response to the medical message 702-6, genomic data system 124 determines, based on the patient information 740, whether the patient 750 was previously sequenced. When the patient 750 was previously sequenced, genomic data system 124 sends a medical message 702-7 to EHR system 142 with a prompt 742 to reuse sequencing data 126 for the patient 750. In FIG. 16, for example, the GUI may display a genetic testing screen 1604 indicating that sequencing data 126 was found for the patient 750, and providing a selection option to re-use the sequencing data 126 existing for the patient 750 in a genomics test(s).

When the physician selects “RE-USE” in FIG. 16, the GUI may display a genetic testing screen 1704 allowing the physician to enter one or more procedure codes 720 for the desired genetic test(s). In response to the input from the physician, EHR system 142 sends an HL7 message 730 to a data translation application 760 containing a procedure code(s) 720 (see FIG. 18B). Data translation application 760 converts or translates the HL7 message 730 into a JSON file 736 or another type of message or file, and sends the JSON file 736 containing the procedure code(s) 720 to genomic data system 124. In response, genomic data system 124 initiates genetic testing on the sequencing data 126 for the patient 750. To do so, genomic data system 124 parses the JSON file 736 to extract the procedure code(s) 720 indicating the genetic test requested or ordered for the patient 750, selects a workflow 502 to analyze the sequencing data 126 based on the procedure code(s) 720, and initiates the selected workflow 502 to analyze the sequencing data 126 for the patient 750 and generate test results 212. Genomic data system 124 inserts the test results 212 in a JSON file 736, and sends the JSON file 736 to data translation application 760. In turn, data translation application 760 converts or translates the JSON file 736 into an HL7 message 730 that is sent to the EHR system 142. The physician may then review the test results 212, such as on requestor client 130.

When sequencing data 126 is not found for the patient 750, genomic data system 124 may initiate sample collection for the patient 750 as described above. When a biological sample for the patient 750 is received, a sequencing process is performed at laboratory 122 using sequencing equipment 120, and the raw sequence data 208 is analyzed to generate the test results 212. In general, laboratory procedures related to genetics may include accessioning, sample plating, storage, extraction, library preparation, enrichment, and sequencing processes. These processes acquire genetic material from a sample, separate the genetic material from other constituents, duplicate the genetic material, and quantify the genetic material order to determine a swathe of sequence data, such as an exome or entire genome for a subject (e.g., a human, an animal, a pathogen, an organelle, etc.).

Sequencing may be performed according to any of a variety of techniques, including short-read and long-read techniques. In one embodiment, the sequencing is performed as Sequencing by Synthesis (SBS) at genetic analyzer equipment. For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers. In one embodiment, the chemical identifiers comprise nucleotide sequences that are detectable during the sequencing process to determine a corresponding Laboratory Sample Identifier (LSI).

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

Sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence which can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome, or another set of genomic data, is being tested, different reagents (e.g., probes, primers, etc.) may be chosen. That is, different reagents may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

In some embodiments, genetic material is used for detection of a pathogen rather than for sequencing. Detecting a pathogen may involve the use of a real-time Polymerase Chain Reaction (PCR) system that performs PCR. The real-time PCR system may further add a reactive agent to individual wells of a library preparation microplate, that fluoresces when bound to genetic material for the pathogen. By analyzing fluorescence at known periods of time after PCR has initiated, presence of a pathogen is determined. Genetic testing for a pathogen may thereby forego sequencing in some embodiments.

Raw sequence data generated during synthesis may be stored in a file format, such as Binary Base Call (BCL), depending on the sequencing equipment used. This raw data may be fed to an analytical pipeline, such as a cloud-based computing environment. Raw sequence data may be processed by the analytical pipeline into a second format, such as a text-based FASTQ format, that reports the sequence information (i.e., the sequence reads) and corresponding quality scores. The second format is then analyzed to perform alignment of sequence reads to a reference genome, such as a reference genome reported in a Browser Extensible Data (BED) file. The aligned sequence data may be reported as a Binary Alignment Map (BAM) file. The aligned sequence data may then be called, resulting in a Variant Call Format (VCF) file reporting called variants at each location of the genome that was sequenced, together with secondary metrics, such as quality indicator metrics.

The called sequence data may be provided to a data analyst via a User Interface (UI), such as a GUI presented via a display. The technician may then validate the resulting called sequence data and release it for reporting to subjects, health care providers, and/or scientists.

Although specific embodiments were described herein, the scope of the invention is not limited to those specific embodiments. The scope of the invention is defined by the following claims and any equivalents thereof.

What is claimed is:

1. A system, comprising:
a data repository configured to store sequencing data for sequencing participants generated based on genomic sequencing; and
a test manager, comprising a processor and memory, configured to execute an algorithm to:
receive one or more incoming medical messages from a requestor regarding a patient;
determine whether the data repository stores sequencing data for the patient;
when the data repository stores the sequencing data for the patient:
parse an incoming medical message of the incoming medical messages to identify a procedure code indicating a genetic test requested for the patient;
process the procedure code to select a workflow to analyze the sequencing data with one or more analysis tools for the genetic test; and
initiate the workflow to analyze the sequencing data for the patient and generate test results; and
provide the test results for the patient to the requestor.

2. The system of claim 1, further comprising:
sequencing equipment configured to perform the genomic sequencing on samples for the sequencing participants.

3. The system of claim 2, wherein:
when the data repository does not store the sequencing data for the patient:
the test manager is further configured to execute the algorithm to provide an outgoing medical message to the requestor with information regarding collection of a sample from the patient;
the sequencing equipment is further configured to perform the genomic sequencing on the sample for the patient when received to generate raw sequence data for the patient; and
the test manager is further configured to execute the algorithm to initiate the workflow to analyze the raw sequence data for the patient and generate the test results.

4. The system of claim 1, wherein:
the test manager is further configured to execute the algorithm to:
provide, in response to at least one of the incoming medical messages, an outgoing medical message to the requestor indicating whether the sequencing data is stored for the patient.

5. The system of claim 1, wherein:
at least one of the incoming medical messages comprises a JavaScript Object Notation file; and
the test manager is further configured to execute the algorithm to parse the JavaScript Object Notation file to identify the procedure code indicating the genetic test requested for the patient.

6. The system of claim 1, wherein:
at least one of the incoming medical messages comprises Health Level-7 message; and
the test manager is further configured to execute the algorithm to parse the Health Level-7 message to identify the procedure code indicating the genetic test requested for the patient.

7. The system of claim 1, wherein:
the test manager is further configured to execute the algorithm to store a code mapping of a plurality of procedure codes to a plurality of workflows, and to select the workflow from the plurality of workflows based on the procedure code and the code mapping.

8. The system of claim 1, wherein:
the test manager is further configured to execute the algorithm to perform a classification process to classify a status of the patient, wherein the status is selected from one or more of:
an unconsented status where a patient has no record of consent to sequencing;
a consented status where a patient has the record of consent, but no sample has been received for sampling;
an ordered status where a patient has received a kit for sampling;
a sampled status where a sample for a patient has been received;
a sequenced status where sequencing data has been generated for a patient;
an analyzed status where sequencing data of a patient has been analyzed; and
a results status where test results have been prepared for a patient.

9. The system of claim 1, wherein:
the test manager is further configured to execute the algorithm to:
accumulate the one or more incoming medical messages over a time period comprising a plurality of procedure codes for the patient;
parse the accumulated incoming medical messages to extract the plurality of procedure codes;
aggregate multiple procedure codes of the plurality of procedure codes that are mapped to a same workflow; and
initiate the same workflow to analyze the sequencing data for the patient based on the aggregated procedure codes.

10. A method, comprising:
storing sequencing data for sequencing participants generated based on genomic sequencing;
receiving one or more incoming medical messages from a requestor regarding a patient; and
determining whether sequencing data is stored for the patient;
when the sequencing data is stored for the patient, the method further comprises:
parsing an incoming medical message of the incoming medical messages to identify a procedure code indicating a genetic test requested for the patient;
processing the procedure code to select a workflow to analyze the sequencing data with one or more analysis tools for the genetic test; and
initiating the workflow to analyze the sequencing data for the patient and generate test results;
wherein the method further comprises providing the test results for the patient to the requestor.

11. The method of claim 10, further comprising:
performing the genomic sequencing on samples for the sequencing participants with sequencing equipment.

12. The method of claim 11, wherein:
when the sequencing data is not stored for the patient, the method further comprises:
providing an outgoing medical message to the requestor with information regarding collection of a sample from the patient;
performing the genomic sequencing on the sample for the patient with the sequencing equipment when received to generate raw sequence data for the patient; and
initiating the workflow to analyze the raw sequence data for the patient and generate the test results.

13. The method of claim 10, further comprising:
providing, in response to at least one of the incoming medical messages, an outgoing medical message to the requestor indicating whether the sequencing data is stored for the patient.

14. The method of claim 10, wherein:
at least one of the incoming medical messages comprises a JavaScript Object Notation file; and
the parsing the incoming medical message comprises parsing the JavaScript Object Notation file to identify the procedure code indicating the genetic test requested for the patient.

15. The method of claim 10, wherein:
at least one of the incoming medical messages comprises Health Level-7 message; and
the parsing the incoming medical message comprises parsing the Health Level-7 message to identify the procedure code indicating the genetic test requested for the patient.

16. The method of claim 10, further comprising:
storing a code mapping of a plurality of procedure codes to a plurality of workflows;
wherein the selecting the workflow comprises selecting the workflow from the plurality of workflows based on the procedure code and the code mapping.

17. The method of claim 10, further comprising:
performing a classification process to classify a status of the patient, wherein the status is selected from one or more of:
an unconsented status where a patient has no record of consent to sequencing;
a consented status where a patient has the record of consent, but no sample has been received for sampling;
an ordered status where a patient has received a kit for sampling;
a sampled status where a sample for a patient has been received;
a sequenced status where sequencing data has been generated for a patient;
an analyzed status where sequencing data of a patient has been analyzed; and
a results status where test results have been prepared for a patient.

18. The method of claim 10, further comprising:
accumulating the one or more incoming medical messages over a time period comprising a plurality of procedure codes for the patient;
parsing the accumulated incoming medical messages to extract the plurality of procedure codes;
aggregating multiple procedure codes of the plurality of procedure codes that are mapped to a same workflow; and
initiating the same workflow to analyze the sequencing data for the patient based on the aggregated procedure codes.

19. A non-transitory computer readable medium embodying programmed instructions executed by a processor, wherein the instructions direct the processor to implement a method comprising:
storing sequencing data for sequencing participants generated based on genomic sequencing;
receiving one or more incoming medical messages from a requestor regarding a patient; and
determining whether sequencing data is stored for the patient;
when the sequencing data is stored for the patient, the method further comprises:
parsing an incoming medical message of the incoming medical messages to identify a procedure code indicating a genetic test requested for the patient;
processing the procedure code to select a workflow to analyze the sequencing data with one or more analysis tools for the genetic test; and
initiating the workflow to analyze the sequencing data for the patient and generate test results;
wherein the method further comprises providing the test results for the patient to the requestor.

20. The computer readable medium of claim 19, wherein:
when the sequencing data is not stored for the patient, the method further comprises:
providing an outgoing medical message to the requestor with information regarding collection of a sample from the patient;
performing the genomic sequencing on the sample for the patient with sequencing equipment when received to generate raw sequence data for the patient; and
initiating the workflow to analyze the raw sequence data for the patient and generate the test results.
